(19) United States
(12) Patent Application Publication
(10) Pub. No.: US 2021/0186989 A1
(43) Pub. Date: Jun. 24, 2021

(54) ENHANCED TRAINED IMMUNITY IN MYELOID CELLS BY SHIP-1 INHIBITION

(71) Applicants: CENTRO NACIONAL DE INVESTIGACIONES CARDIOVASCULARES CARLOS III, Madrid (ES); SYRACUSE UNIVERSITY, New York, NY (US); THE RESEARCH FOUNDATION FOR THE STATE UNIVERSITY OF NEW YORK, New York, NY (US)

(72) Inventors: David SANCHO MADRID, Madrid (ES); Paula SAZ LEAL, Madrid (ES); Carlos DE FRESNO SÁNCHEZ, Madrid (ES); John CHISHOLM, Fayetteville, NY (US); William KERR, Syracuse, NY (US)

(21) Appl. No.: 16/972,422

(22) PCT Filed: Jun. 6, 2019
(86) PCT No.: PCT/EP2019/064871

§ 371 Date: Dec. 4, 2020

(30) Foreign Application Priority Data
Jun. 6, 2018 (EP) 18382404.4

(51) Int. Cl. A61K 31/575; A61K 31/568; A61K 45/06; A61P 37/04

(52) U.S. Cl. CPC A61K 31/575; A61P 37/04; A61K 45/06; A61K 31/568

(57) ABSTRACT

The present invention refers to the medical field. Particularly, it refers to SHIP-1 inhibitors for use in enhancing the non-specific response of trained innate immune cells (i.e. enhancing the training of the innate immune cells) in a subject, wherein the SHIP-1 inhibitor is administered before, after or simultaneously to a treatment with a stimulus responsible for training the innate immune cells.

### FIELD OF THE INVENTION

The present invention refers to the medical field. Particularly, it refers to SHIP-1 inhibitors for use in enhancing the non-specific response of trained innate immune cells (i.e. enhancing the training of the innate immune cells) in a subject, wherein the SHIP-1 inhibitor is administered before, after or simultaneously to a treatment with a stimulus responsible for training the innate immune cells.

### STATE OF THE ART

According to the established prior art, the general view that only adaptive immunity can build immunological memory has been challenged. In organisms lacking adaptive immunity, as well as in mammals, the innate immune system can mount resistance to reinfection, a phenomenon termed “trained immunity” or “innate immune memory.” Consequently, trained immunity can be defined as a de-facto innate immune memory that induces enhanced inflammatory and antimicrobial properties in innate immune cells, responsible for an increased non-specific response to subsequent infections and improved survival of the host. Trained immunity is orchestrated by epigenetic reprogramming, broadly defined as sustained changes in gene expression and cell physiology that do not involve permanent genetic changes such as mutations and recombination, which are essential for adaptive immunity. The discovery of trained immunity may open the door for novel vaccine approaches, new therapeutic strategies for the treatment of immune deficiency states, and modulation of exaggerated inflammation in autoinflammatory diseases.

Thus, innate immune cells challenged with certain stimuli undergo long-lasting changes that result in improved response to a second challenge by the same or even different microbial insults. Stimuli driving trained immunity lead to a deep metabolic change with a noted shift from oxidative phosphorylation towards aerobic glycolysis. Moreover, this initial activation is accompanied by sustained changes in the epigenome, mainly via histone methylation and acetylation. Hematopoietic stem cell reprogramming supports the long-lasting effect of trained immunity.

It has been established that among the stimuli inducing trained immunity, exposure to a low dose of Candida albicans or the fungal cell wall component beta-glucan protects mice from secondary lethal systemic candidiasis or heterologous Staphylococcus aureus septicemia. This acquired resistance does not rely on TB lymphocytes or NK cells but occurs in a myeloid-dependent manner. Mechanistically, the C-type lectin receptor Dectin-1 is critical for the sensing of Candida albicans or beta-glucan, leading to immune training of monocytes/macrophages. These primed macrophages show heightened production of proinflammatory cytokines such as TNF-alpha to a wide variety of insults. This Dectin-1-mediated training, which also includes glycolytic switch and epigenetic rewiring, relies on activation of the PI3K (Phosphoinositide 3-kinase)/mTOR (mammalian target of rapamycin)/HIF-1 alpha (hypoxia-inducible factor-1α) pathway.

Departing from the previous knowledge that innate immune cells can be trained to exhibit an enhanced and lasting response to subsequent infections with microbial components, the present invention is focused on solving a specific technical problem which is how to further improve the non-specific response of trained innate immune cells thus providing an improved prophylactic treatment or prevention of subsequent infections. The present invention solves this problem by demonstrating that trained immunity in myeloid cells can be further enhanced by means of the use, along with the training of the innate immune cells, of compounds acting as enhancers which are directed to the inhibition of a specific target.

### DESCRIPTION OF THE INVENTION

### Brief Description of the Invention

The present invention refers to the use of SHIP-1 inhibitors (SHIPi) in the non-specific prophylactic treatment or prevention of subsequent infections (causing infectious diseases), by means of the enhancement of the memory and non-specific response of trained innate immune cells; wherein the SHIP-1 inhibitor is administered before, after or simultaneously to a treatment with a stimulus responsible for training the innate immune cells. Particular details of SHIP-1 can be found in any of the following data bases: HGNC: 6079, Entrez Gene: 3635, Ensembl: ENSG00000168918, OMIM: 601582, UniProtKB: Q92835.

As proof of concept, the inventors of the present invention demonstrate that beta-glucan-trained macrophages from mice with a specific SHIP-1 deletion in the myeloid compartment (LysMASHIP-1) showed enhanced TNFα production in response to lipopolysaccharides (LPS). Following β-glucan training, SHIP-1-deficient macrophages exhibited increased phosphorylation of Akt and mTOR targets, correlating with augmented glycolytic metabolism. Enhanced training in the absence of SHIP-1 was histone methyltransferase-dependent, suggesting the involvement of epigenetic reprogramming. Trained LysMASHIP-1 mice showed increased LPS-induced TNFα production in vivo and better protection against infection with Candida albicans compared with control littermates.

Thus, SHIP-1 has a regulatory role in β-glucan-induced training in vitro, affecting all hallmarks involved in that process. Moreover, as cited above, the present invention shows that in vivo SHIP-1 deficiency in the myeloid compartment improves protection conferred by trained immunity. Notably, enhanced pro-inflammatory cytokine production and better protection was achieved in the present invention by pharmacological SHIP-1 inhibition both in mice and human peripheral blood mononuclear cells (PBMCs), providing a potential therapeutic approach to boost trained immunity.

Particularly, in the present invention, both chemical (for example mice treated with 3α-aminocholestane; 3AC; SHIPi) and genetic means (for example mice with a specific SHIP-1 deletion in the myeloid compartment [LysMASHIP-1], small hairpin/siRNA, microRNAs, also gene editing) have been used for achieving the inhibition of SHIP-1. On the other hand, beta-glucan or low doses of C. albicans have been used in the present invention as an example of stimulus conferring a first stimulus responsible for reprogramming the immune response, thus training innate immune cells. A lethal dose of C. albicans or LPS have been used in the present invention in order to determine the survival rate and inflammatory response, respectively, in four different types of animal models.

Such as it is shown in FIG. 3 and FIG. 4, wild type (WT) mice, and non-trained mice having SHIP-1 inhibited (or depleted in the myeloid compartment), rapidly succumbed upon receiving the lethal dose of C. albicans. As expected, WT mice trained with beta-glucan or with a low dose of C. albicans extended their lifespan for a longer period. Surprisingly, animal models having SHIP-1 inhibited and trained with beta-glucan or with a low dose of C. albicans, showed an unexpected increased survival rate. Therefore, it can be concluded that the response of trained innate immune cells to subsequent infections, can be surprisingly boosted by the inhibition of SHIP-1. Interestingly, according to the results provided in FIG. 3 and FIG. 4, when SHIPi is administered or SHIP-1 is mutated in non-trained mice, these mice rapidly die thus suggesting that the inhibition of SHIP-1, by itself, do not confer any improved effect over non-trained innate immune cells. In other words, the present invention shows a synergistic effect which is observed when both SHIPi and a stimulus responsible for training innate immune cells are combined. This synergistic effect is unexpected mainly considering that, as explained above, the present invention indicates that, in our context, SHIPi has no role in (non-trained) innate immunity to infection but, in contrast, the inhibition of SHIP-1 enhances the memory and non-specific response of trained innate immune cells. Thus, the present invention shows that SHIP-1 deletion in myeloid cells, following β-glucan training, augments Akt activation and glycolysis switch, resulting in enhanced trained immunity both in vitro and in vivo. SHIP-1-deficient macrophages showed increased basal Akt phosphorylation. Akt overactivation is associated with a survival advantage, which however did not significantly impact glycolysis or response to LPS challenge in non-trained LysMASHIP-1 BMDMs compared to WT. Moreover, β-glucan training resulted in increased recovery of WT BMDMs, attributable to enhanced survival, but it did not further enhance survival or proliferation in SHIP-1-deficient BMDMs. Instead, β-glucan training led to a superior intrinsic TNFα production in response to LPS, higher upon SHIP-1 deficiency. This SHIP-1-mediated effect was largely Syk-dependent and Raf-1 independent, supporting a role of the phosphatase through this pathway.

Consequently, SHIP-1 is herein defined as a new target to improve β-glucan-induced myeloid-dependent trained immunity. Additionally, a pharmacological approach to take advantage of this new mechanism, namely the SHIP-1 inhibitor 3AC, is provided by the present invention. Considering that germline-deficient SHIP-1 mice display gross inflammatory abnormalities, 3AC administration in vivo has to be tightly regulated. In this regard, a pulsatile but not extended dosing strategy of 3AC was effective in boosting β-glucan-induced resistance to Candida infection. Moreover, 3AC administration expands the hematopoietic stem cell compartment. Since modulation of myeloid progenitors in the bone marrow is an integral component of trained immunity, SHIP-1 inhibition could influence this compartment. In this regard, transfer of hematolymphoid (spleen and bone marrow) cells from tumor-challenged, 3AC-treated, long-term surviving mice protected naïve recipients to tumor challenge. Although pulsatile inhibition of SHIP-1 enhances NK and T cell anti-tumor effector function, it is feasible that SHIP-1 inhibition could have also affected bone marrow progenitors to promote training. Consequently, the present invention proposes a strategy to improve trained immunity since it demonstrates that SHIP-1 inhibition potentiates the canonical trained immunity pathway, and boosts a long-lasting effect also appreciable in vivo. Since PI3K/Akt pathway activation is critical for trained immunity not only in response to β-glucan but also to other stimuli such as the Bacillus Calmette-Guérin (BCG) vaccine, SHIP-1 inhibition could represent a broad strategy to boost trained immunity. In this regard, BCG-induced upregulation of the microRNA-155 has proved to repress SHIP-1 induction, modulating ROS production in macrophages. Indeed, SHIP-1 displays an inhibitory function in NOD2 signaling, the BCG-mediated trained immunity pathway. Considering that BCG vaccination confers cross-protection to human viral infections, SHIP-1 inhibitor could improve the protective effect of BCG. Altogether, as a proof of concept, our data indicate that the trained immunity process can be boosted. Moreover, SHIP-1 inhibitors are herein proposed as potential pharmacological tools to improve trained immunity in clinical settings where enhancement of inflammatory responses is beneficial, such as infections.

So, the first embodiment of the present invention refers to SHIP-1 inhibitors for use in enhancing the non-specific response of trained innate immune cells (i.e. enhancing the training of the innate immune cells) in a subject, wherein the SHIP-1 inhibitor is administered before, after or simultaneously to a treatment with a stimulus responsible for training the innate immune cells.

In a preferred embodiment, the present invention refers to SHIP-1 inhibitors for use in the non-specific prophylactic treatment or prevention of infectious diseases, wherein the SHIP-1 inhibitor is administered before, after or simultaneously to a treatment with a stimulus responsible for training the innate immune cells.

In a preferred embodiment, the present invention refers to SHIP-1 inhibitors for use in the non-specific prophylactic treatment or prevention of subsequent infections (second or further infections) caused either by the same or different microorganisms, wherein the SHIP-1 inhibitor is administered before, after or simultaneously to a treatment with a pathogenic microorganism or any part thereof responsible for training the innate immune cells.

The second embodiment of the present invention refers to a combination drug product comprising a SHIP-1 inhibitor, a stimulus responsible for training the innate immune cells and optionally pharmaceutically acceptable carriers.

The third embodiment of the present invention refers to a pharmaceutical composition comprising the above cited combination drug product and optionally pharmaceutically acceptable carriers. In a preferred embodiment, the pharmaceutical composition is a vaccine.

The fourth embodiment of the present invention refers to SHIP-1 for use in the non-specific prophylactic treatment or prevention of subsequent infections causing infectious diseases by means of the enhancement of the non-specific response of trained innate immune cells; before, after or simultaneously to a treatment with a stimulus responsible for training the innate immune cells, characterized in that SHIP-1 expression is inhibited or interrupted.

The fifth embodiment of the present invention refers to a method for enhancing the non-specific response of trained innate immune cells (i.e. enhancing the training of the innate immune cells) in a subject wherein the SHIP-1 inhibitor is administered before, after or simultaneously to a treatment with a stimulus responsible for training the innate immune cells.

The sixth embodiment of the present invention refers to a method for the non-specific prophylactic treatment or prevention of infectious diseases, wherein the SHIP-1 inhibitor is administered before, after or simultaneously to a treatment with a stimulus responsible for training the innate immune cells.

The seventh embodiment of the present invention refers to a method for the non-specific prophylactic treatment or prevention of subsequent infections (second or further infections) caused either by the same or different microorganisms, wherein the SHIP-1 inhibitor is administered before, after or simultaneously to a treatment with a pathogenic microorganism or any part thereof responsible for training the innate immune cells.

In a preferred embodiment, the present invention is not limited to a specific SHIP-1 inhibitor because what the inventors of the present invention have surprisingly shown (as demonstrated in Example 13 wherein a LysMASHIP-1 mice is assayed) is that it is the inhibition of SHIP-1 (irrespective of the type of inhibitor or the means used for the inhibition of SHIP-1) what can be used for enhancing the memory and non-specific response of trained innate immune cells. Thus, SHIP-1 is defined in the present invention as a therapeutic target whose inhibition would result in improving the health of patients. By way of example, the SHIP-1 inhibitor to be used in the present invention could specifically target SHIP-1 gene and inhibit its translation, or they could be an antagonist selective for SHIP-1. Consequently, in a preferred embodiment of the invention, genetic means are used for inhibiting SHIP-1 expression. By way of example, the PCT application WO2003053341 (which is herein incorporated by reference in its entirety) discloses a variety of antisense oligonucleotides, which are targeted to a nucleic acid encoding Ship-1, and which modulate the expression of Ship-1.

Alternatively, by way of example, the present invention can be also implemented using SHIP-1 inhibitors. Among the SHIP-1 inhibitors that can be used in the present invention are those of formula (I), and pharmaceutically acceptable salts thereof, disclosed in the patent application US20130102577, which is herein included by reference in its entirety, particularly those SHIP-1 inhibitors disclosed in Examples 1 to 18 of US20130102577. In a preferred embodiment, the SHIP-1 inhibitor is 3α-aminocholestane (3AC). However, other examples of SHIP-1 inhibitors that could be used in the present invention are tryptamine-based SHIP inhibitors as disclosed in the patent application US20170189380 which is included herein by reference in its entirety. Alternatively, pan-SHIP inhibitors 1PIE, 2PIQ and 6PTQ as depicted in FIG. 5 of [Fuhler G M et al., 2012. Therapeutic potential of SH2 domain-containing inositol-5′-phosphatase 1 (SHIP]) and SHIP2 inhibition in cancer. Mol Med. 2012 Feb. 10; 18:65-75] can be used in the present invention. On the other hand, quinoline-based SHIP inhibitors can be used in the present invention, preferably NSC13480 and NSC305787 as depicted in FIG. 2 of [Russo C M et al., 2015. Synthesis and initial evaluation of quinoline-based inhibitors of the SH2-containing inositol 5′-phosphatase (SHIP). Bioorg Med Chem Lett. 2015 Nov. 15; 25(22):5344-8].

In a preferred embodiment the SHIP-1 inhibitor is administered following any suitable route of administration, preferably intravenous administration, intraperitoneal administration, intramuscular administration, subcutaneous administration, etc. On the other hand, SHIP-1 inhibitors can be also administered following other modes of administration, for example: oral, nasal, rectal, etc.

In a preferred embodiment, the infectious disease which would be prevented by implementing the present invention is an infectious disease caused by Gram negative or Gram positive bacteria, viruses, fungi or parasites. Of note, in the present invention, beta-glucan-induced trained immunity in the absence of SHIP-1 not only improved protection to C. albicans (FIG. 3C), but also increased pro-inflammatory cytokine production following challenge with systemic lipopolysaccharide (LPS, FIG. 3B). LPS comes from the cell wall of gram-negative bacteria and therefore, it constitutes a model of inflammation induced by this kind of pathogens. This data suggest that SHIP-1 also regulates trained immunity in response to bacterial infections, preferably Gram negative bacteria.

In a preferred embodiment, the present invention is not limited to a specific stimulus responsible for training the innate immune cells, because what the inventors of the present invention have surprisingly demonstrated is that it is the inhibition of SHIP-1 in trained innate immune cells (irrespective of the method or stimulus used for implementing said training) what can be used for enhancing the memory and non-specific response of trained innate immune cells. In a preferred embodiment, the stimulus responsible for training the innate immune cells can be, among others, Plasmodium falciparum responsible for causing malaria [Jacob E. Schrum et al., 2018. Cutting Edge: Plasmodium falciparum Induces Trained Innate Immunity. J. Immunol. 2018 Feb. 15; 200(4):1243-1248], fungal chitin [Rizzeto et al., 2016. Fungal Chitin Induces Trained Immunity in Human Monocytes during Cross-talk of the Host with Saccharomyces cerevisiae. J Biol Chem. 2016 Apr. 8; 291(15):7961-72], Pam3Cys, poly-I:C, flagelin, MDP (muramyl dipeptide), TriDAP, oxLDL, Uric Acid, Fumarate, Mevalonate, GM-CSF/IL-3, IL-1beta, IGF1, LPS, beta-glucan, Saccharomyces cerevisiae, low dose of C. albicans or Bacillus Calmette-Guérin (BCG) [Mihai G. Netea et al., 2016. Trained immunity: A program of innate immune memory in health and disease. Science 22 Apr. 2016. Vol. 352, Issue 6284, aaf1098] [Leentjens J. et al., 2018. Trained Innate Immunity as a Novel Mechanism Linking Infection and the Development of Atherosclerosis. Circ Res. 2018 Mar. 2; 122(5):664-669]. Different types/formulations of beta glucans may be used as disclosed for example in [Walachowski S, et al. Molecular Analysis of a Short-term Model of β-Glucans-Trained Immunity Highlights the Accessory Contribution of GM-CSF in Priming Mouse Macrophages Response. Front Immunol. 2017]. In a preferred embodiment, the beta-glucan is beta-1,3(d)-glucan derived from Saccharomyces cerevisiae.

Particularly, the present invention refers to SHIP-1 inhibitor, characterized by the Formula (I), or any salt thereof,

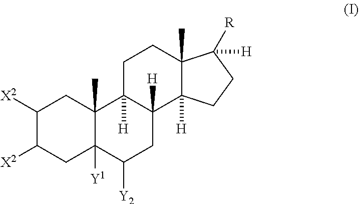

wherein,

X₁ is an amine,

X₂ can be H or OH or amine,

R is a C₁-C₁₁ alkyl,

Y₁ can be H or OH,

Y₂ can be H or OH,

or a molecule able to specifically target SHIP-1 gene and inhibit its translation, for use in the non-specific prophylactic treatment or prevention of infectious diseases, wherein the SHIP-1 inhibitor is administered before, after or simultaneously to a treatment with a pathogenic microorganism or any part thereof which causes a stimulus responsible for training the innate immune cells.

The present invention also refers to a combination drug product comprising a SHIP-1 inhibitor characterized by the Formula (I), or any salt thereof,

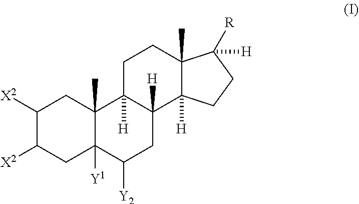

wherein,

X₁ is an amine,

X₂ can be H or OH or amine,

R is a C₁-C₁₁ alkyl,

Y₁ can be H or OH,

Y₂ can be H or OH,

a pathogenic microorganism or any part thereof which causes a stimulus responsible for training the innate immune cells and optionally pharmaceutically acceptable carriers.

The present invention also refers to a pharmaceutical composition comprising the combination drug product and optionally pharmaceutically acceptable carriers.

In a preferred embodiment the pharmaceutical composition is a vaccine composition comprising the combination drug product.

In a preferred embodiment the SHIP-1 inhibitor is selected from:

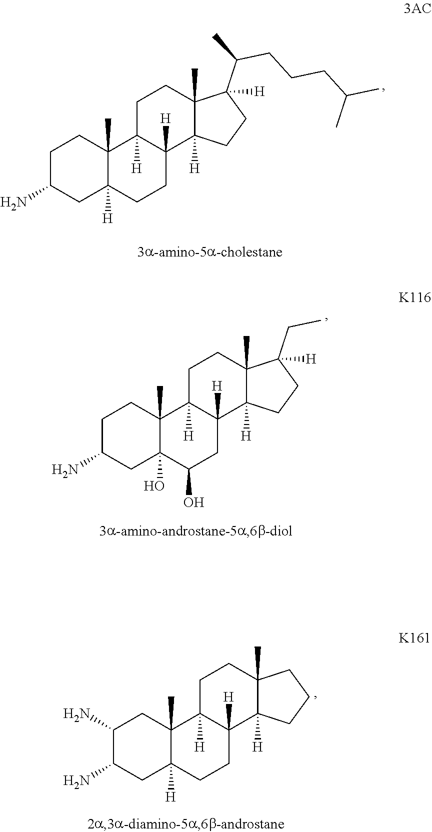

In a preferred embodiment the SHIP-1 inhibitor is 3α-aminocholestane (3AC).

In a preferred embodiment the SHIP-1 inhibitor specifically targets SHIP-1 gene and inhibits its translation, or it is an antagonist selective for SHIP-1.

In a preferred embodiment the infectious disease is caused by an infection with a Gram negative or Gram positive bacteria, viruses, fungi or parasites.

In a preferred embodiment the pathogenic microorganism or any part thereof which causes a stimulus responsible for training the innate immune cells is beta-glucan or Candida albicans, preferably a low dose of Candida albicans (approximately 4×10\hat{ }5 cfu/Kg).

In a preferred embodiment the beta-glucan is beta-1,3(d)-glucan, preferably derived from Saccharomyces cerevisiae.

For the purpose of the present invention the following definitions are given:
The expression “trained innate immunity” or “trained innate immune cells” refers to a de facto innate immune memory that induces enhanced inflammatory and antimicrobial properties in innate immune cells, responsible for an increased non-specific response to subsequent infections and improved survival of the host. “Trained innate immunity” is achieved by applying “stimuli” responsible for training the innate immune cells” which undergoes long-lasting changes that result in improved response to a second challenge by the same or even different microbial insults. Trained innate immune cells are characterized by an enhanced pro-inflammatory cytokine production.
The expression “enhancing the non-specific response of trained innate immune cells” refer to a situation where the non-specific response of trained innate immune cells is boosted or improved by means of the inhibition of SHIP-1, as compared with the non-specific response of trained innate immune cells when SHIP-1 is not inhibited. Said boosted or improved non-specific response of the trained innate immune cells is characterized by, for example, an increased production of pro-inflammatory cytokines in macrophages, increased phosphorylation of Akt and/or mTor targets in macrophages, increased pro-inflammatory cytokine production in vivo upon LPS or any other challenge and improved protection against infection (increased survival rate following lethal C. albicans infection or any other pathogenic microogranism). In a preferred embodiment, said boosted or improved non-specific response of the trained innate immune cells is characterized by an increased production of cytokines, preferably TNF-alpha, wherein the cytokine production when the SHIP-1 inhibitor is administered along with stimuli responsible for training the innate immune cells is at least 10%, 20%, 30%, 40%, 50%, 60%, 70%, 80%, 90% or 100% higher than the cytokine production when SHIP-1 inhibitor is not administered along with stimuli responsible for training the innate immune cells.
The expression “stimulus responsible for training the innate immune cells” refers to any molecule, non-pathogenic or pathogenic microorganism, or any part thereof, able to induce “trained innate immunity” in innate immune cells. It can be, for example, beta-glucan or C. albicans, preferably a low dose of C. albicans. However, as cited above, other described inducers can be used. In a preferred embodiment, the beta-glucan is beta-1,3(d)-glucan derived from Saccharomyces cerevisiae.
The expression “pathogenic microorganism” refers to any microorganism capable of injuring its host, e.g., by competing with it for metabolic resources, destroying its cells or tissues, or secreting toxins. The injurious microorganisms include viruses, bacteria, mycobacteria, fungi, protozoa, and some helminths.
The expression “infectious diseases” refers to a disease caused by an infection with Gram negative or Gram positive bacteria, viruses, fungi or parasites.
The expression “subsequent infections” or “subsequent infectious diseases” refers to a second or further infection or infectious disease caused either by the same or different microorganism when a microbe (e.g. Candida) is used for training innate immune cells, after recovery from or during the course of a primary infection. In the case of a non-infectious stimulus inducing training, it will improve response to primary infections.
The expression “non-specific response” or “non-specific prophylactic treatment or prevention” means that the response or prophylactic treatment or prevention is achieved by the innate immune system, thus protecting the patient from any challenge (comprising Gram negative or Gram positive bacteria, viruses, fungi or parasites), irrespective of the stimulus used for training the innate immune cells.
The term “comprising” it is meant including, but not limited to, whatever follows the word “comprising”. Thus, use of the term “comprising” indicates that the listed elements are required or mandatory, but that other elements are optional and may or may not be present.
By “consisting of” is meant including, and limited to, whatever follows the phrase “consisting of”. Thus, the phrase “consisting of” indicates that the listed elements are required or mandatory, and that no other elements may be present.

### DESCRIPTION OF THE FIGURES

FIG. 1. SHIP-1 deletion boosts beta-glucan-induced trained immunity in macrophages. (A) SHIP-1 expression by Western Blot, normalized to beta-Actin, in bone marrow macrophages (BMDMs) exposed (+) or not (−) to beta-glucan (whole glucan particles) for the indicated time. Representative experiment of three performed. (B) SHIP-1 protein expression in WT and LysMASHIP-1 BMDMs. Representative experiment of six performed. (C) In vitro model to test trained immunity in mouse BMDMs. (D) Dectin-1 expression in WT and LysMASHIP-1 BMDMs before beta-glucan training according to model in FIG. 1C. FACS histograms representative of four independent experiments. (E) TLR4 expression in WT and LysMASHIP-1 BMDMs both under non-trained (left panel) or beta-glucan primed (right panel) conditions, just before LPS stimulation according to model in FIG. 1C. FACS histograms representative of four independent experiments. (F) WT and LysMASHIP-1 BMDMs were stimulated (+) or not (−) with beta-glucan or LPS, and TNF-alpha production was analyzed in supernatants according to the model in FIG. 1C. (G) WT and LysMASHIP-1 BMDMs were incubated (+) or not (−) with a Syk inhibitor (R-406, 1.5 μM) or a Raf-1 inhibitor (GW5074, 1 mM) for 30 minutes previous to beta-glucan training and after washing it out. TNF alpha production was analyzed in supernatants after LPS stimulation according to model in FIG. 1C. (F,G) Four (F) and three (G) independent experiments are shown. *p<0.05, **p<0.01, paired Student's t test comparing WT and LysMASHIP-1. (F) #p<0.05, paired Student's t test comparing within the same genotype stimulated or not with β-glucan.

FIG. 2. SHIP-1 regulates molecular and metabolic hallmarks of trained immunity. (A) WT and LysMASHIP-1 BMDMs were exposed to beta-glucan for the indicated time and phospho-Akt, Akt, phospho-S6, phospho-4EBP1 and β-Actin analyzed by WB. Representative experiment of five performed. (B-E) WT and LysMASHIP-1 BMDMs were left untreated (dashed lines) or treated for 1 day with beta-glucan (solid lines), washed, rested for 3 days and re-plated in equal numbers for determination of extracellular acidification rate (ECAR). ECAR in a glycolysis stress test was analyzed upon sequential addition of glucose, oligomycin and 2-deoxyglucose (2DG) as indicated (B). Analysis of basal glycolysis (C), maximal glycolysis (D) and glycolytic reserve (E). (B-E) Mean±SEM and individual data of three independent cultures in a representative experiment of two performed. (F) WT and LysMΔSHIP-1 BMDMs were incubated (+) or not (−) with the methyltransferase inhibitor MTA (500 μM) or the histone demethylase inhibitor Pargyline (6 μM) for 30 minutes previous to beta-glucan training and after washing it out. TNF-alpha production was analyzed in supernatants after LPS stimulation according to model in FIG. 1C. Individual data corresponding to 3 independent experiments are shown. (C-F) *p<0.05, **p<0.01, unpaired (C-E) and paired (F) Student's t test comparing WT and LysMΔSHIP-1. (C-E) #p<0.05, unpaired Student's t test comparing within the same genotype stimulated or not with β-glucan.

FIG. 3. Myeloid-specific deletion of SHIP-1 improves trained immunity in vivo. (A) In vivo model of training by two beta-glucan intraperitoneal (i.p.) injections, indicating secondary challenges and readouts. (B) WT and LysMΔSHIP-1 mice, either beta-glucan-trained (+) or not (−), were i.p. injected with LPS according to model in FIG. 3A. Serum was collected after 1 hour and TNF-alpha analyzed. Individual data and mean±SEM of a representative experiment of two performed is shown. *p<0.05, unpaired Student's t test comparing WT and LysMΔSHIP-1. #p<0.05, unpaired Student's t test comparing the same genotype stimulated or not with β-glucan. (C) WT and LysMΔSHIP-1 mice, either beta-glucan-trained (solid lines) or not (dashed lines), were systemically infected with a lethal dose of Candida albicans. Survival was monitored. A pool of two experiments is shown including between 6 and 16 mice per group as indicated. (D) In vivo model of training by a systemic infection with a low dose of Candida albicans followed by a second lethal challenge with the same pathogen. (E) Survival curve of WT and LysMΔSHIP-1 mice according to model in FIG. 3D. A pool of two experiments is shown including between 7 and 13 mice per group as indicated. (C,E) **p<0.01, Log-rank test between WT and LysMΔSHIP-1 mice. #p<0.05, Log-rank test comparing within the same genotype trained or not with β-glucan (C) or C. albicans (E).

FIG. 4. Pharmacological inhibition of SHIP-1 enhances trained immunity. (A) In vitro experimental model applied to mouse BMDMs, indicating when the SHIP-1 inhibitor (SHIPi) 3-alpha-aminocholestane (3AC) was added. (B) Mouse BMDMs were incubated with the SHIPi at the indicated concentrations. TNF-alpha production was analyzed in supernatants of beta-glucan-trained cells after LPS stimulation according to model in FIG. 4A. Mean+SEM of four independent experiments is shown. **p<0.01 paired Student's t test between SHIPi-treated and non-treated cells. (C) In vivo model of training by a systemic infection with a low dose of Candida albicans in the presence of SHIPi followed by a second lethal challenge with the same pathogen. When indicated, the inhibitor was administered intraperitoneally. (D) Survival curve of 0.3% hydroxypropylcellulose (Control) or SHIPi-treated mice according to model in FIG. 4C. A pool of two experiments is shown including between 10 and 19 mice per group as indicated. **p<0.01, Log-rank test between trained PBS and SHIPi-treated. #p<0.05, Log-rank test comparing within the same treatment trained or not with C. albicans. (E) In vitro experimental model applied to human peripheral blood mononuclear cells (PBMCs) indicating when SHIPi was added. (F) TNFα production was analyzed in supernatants of beta-glucan-trained human PBMCs after LPS stimulation according to model in FIG. 4E. Samples from 7 independent donors are shown. *p<0.05, paired Student's t test.

FIG. 5. Surface expression of Dectin-1 and TLR4 in BMDMs. (A) Dectin-1 surface expression was analyzed by FACS in WT and LysMΔSHIP-1 BMDMs before beta-glucan training. (B) TLR4 surface expression was analyzed by FACS in WT and LysMΔSHIP-1 BMDMs both under non-trained (−) or beta-glucan primed (+) conditions, before LPS stimulation. (A,B) Individual data and mean±SEM from a pool of two experiments is shown including three BMDMs cultures per experiment. Each dot represents an independent cell culture.

FIG. 6. Recovered live BMDMs before LPS stimulation. WT and LysMΔSHIP-1 BMDMs were exposed (+) or not (−) to beta-glucan according to model in FIG. 1C. At day 5 and before LPS stimulation, the number of viable BMDMs was determined by FACS based on Hoechst 33258 exclusion. Individual data from four independent experiments are shown. Significance was assessed by paired Student's t test between genotypes under the same experimental conditions. **p<0.01, paired Student's t test comparing WT and LysMΔSHIP-1. #p<0.05, paired Student's t test comparing within the same genotype stimulated or not with β-glucan.

### DETAILED DESCRIPTION OF THE INVENTION

### Example 1. Mice

Mice, all in C57BL/6 background, were bred at CNIC under specific pathogen-free conditions. Mouse colonies include Wild-type C57BL/6J (WT used for SHIP-1 inhibition experiments), LysM^{+/+}SHIP-1^{flox/flox} (WT) and LysM^{Cre/+}SHIP-1^{flox/flox} (LysMΔSHIP-1) and were kept as littermates. Experiments were conducted with age-matched mice. Experiments were approved by the animal ethics committee at CNIC and conformed to Spanish law under Real Decreto 1201/2005. Animal procedures were also performed in accordance to EU Directive 2010/63EU and Recommendation 2007/526/EC.

### Example 2. Mouse Bone Marrow-Derived Macrophage Differentiation

To obtain mouse bone marrow-derived macrophages (BMDMs) from WT and LysMΔSHIP-1 mice, femurs were collected and flushed, and red blood cells were lysed using RBC Lysis Buffer (Sigma, St. Louis, Mo.) for 3 minutes at room temperature (RT). Cell suspensions were plated in non-treated cell culture plates (Corning, Corning, N.Y.) in RPMI 1640 (Sigma) supplemented with 10% heat-inactivated fetal bovine serum (FBS, Sigma), 1 mM pyruvate (Lonza, Bassel, Switzerland), 100 μM non-essential aminoacids (Thermo Fisher Scientific, Walthman, Mass.), 2 mM L-glutamine, 100 U/ml penicillin, 100 μg/ml streptomycin (all three from Lonza) and 50 μM 2-mercaptoethanol (Merck, Darmstad, Germany), herein called R10, plus M-CSF (30% mycoplasma-free L929 cell supernatant) at 37° C. for 5 days. At day 5, BMDMs were detached in phosphate buffered saline (PBS, Gibco) supplemented with 5 mM EDTA (PBS/EDTA, Life Technologies), counted, plated in R10 at the required concentration and rested overnight before any training.

### Example 3. Peripheral Blood Mononuclear Cells (PBMCs)

Buffy coats from healthy volunteers were obtained from Andalusian Biobank after approval by the local Instituto de Salud Carlos III (ISCIII) Research Ethics Committee (PI 36_2017). PBMCs were isolated by differential centrifugation using Biocoll Separating Solution (Cultek, Madrid, Spain). Cells were washed twice in PBS, resuspended in DMEM (Sigma) supplemented with 10% heat-inactivated FBS, 100 μM non-essential aminoacids, 2 mM L-glutamine, 100 U/ml penicillin, 100 μg/ml streptomycin and 50 μM 2-mercaptoethanol, herein called D10; counted and plated for training.

### Example 4. Candida albicans

Candida albicans (strain SC5314, kindly provided by Prof. C. Gil, Complutense University, Madrid, Spain) was grown on YPD-agar plates (Sigma) at 30° C. for 48h.

### Example 5. Trained Immunity In Vitro Models

Mouse Bone Marrow-Derived Macrophages (BMDMs)

BMDMs (10⁵) were plated in 96-well plates (200-μl final volume, Corning) and stimulated with R10 or 100 μg/ml β-glucan (whole glucan particles, WGP, Biothera, Eagan, Minn.) for 24h. Then, cells were washed and rested 3 days in culture medium. At day 4, BMDMs were washed again and primed with 25 ng/ml IFNγ (BD Biosciences, San Jose, Calif.) for 24h. On day 5, a final wash was performed and cells were stimulated with R10 or 1 μg/ml Escherichia coli LPS (EK, Invivogen, San Diego, Calif.). After 24h, supernatants were collected for TNFα measurement by ELISA (Opteia ELISA kit, BD Biosciences).

When required, BMDMs were pre-incubated for 30 minutes prior to β-glucan training with 1.5 μM Syk inhibitor (R406, Hozel diagnostic, Cologne, Germany), 1 mM Raf-1 inhibitor (GW5074, Sigma), 500 μM MTA (5′-Deoxy-5′-(methylthio)adenosine, Sigma), 6 μM Pargyline (Sigma) or SHIP-1 inhibitor (SHIPi, 3-α-aminocholestane, 3AC, Calbiochem, Darmstad, Germany) at the indicated doses. Inhibitors were also added in the first wash-out, before the resting period.

To assess receptor expression and cell viability, 6·10⁵ BMDMs were plated in non-treated 24-well plates (1200-μl final volume, Corning) and followed the training scheme described above. Dectin-1 expression was evaluated at day 0 prior to β-glucan addition. Cell viability and TLR4 expression were assessed on day 5 before LPS stimulation. At indicated times, cells were collected in PBS/EDTA and stained on ice-cold FACS Buffer (PBS/EDTA plus 3% FBS) for flow cytometry analysis.

For WB assays, 3·10⁶ BMDMs were plated in 6-well plates (3-ml final volume, Corning) and stimulated with R10 or 200 μg/ml β-glucan for given times. This increased concentration of β-glucan was used to maintain the mass:cell ratio used for 96-well plates (TNFα measurement)

To address metabolic status, 3·10⁶ BMDMs were plated in non-treated 6-well plates (3-ml final volume, Corning) and followed the training scheme described above but training with 200 μg/ml β-glucan (keep mass:cell ratio). At day 4, without IFN-γ priming, cells were detached in PBS/EDTA, plated at 10⁵ cells/well in sixtuplicates and rested overnight in R10 prior to the Seahorse XF glycolysis stress test (Agilent Technologies).

Human Peripheral Blood Mononuclear Cells (PBMCs)

Total PBMCs (5·10⁵) were plated in 96-well plates (200-μl final volume) and stimulated with 100 μg/ml β-glucan for 24h. Then, cells were washed and rested 6 days in culture medium. At day 7, PBMCs were stimulated with 1 μg/ml LPS (EK). After 24h, supernatants were collected for TNFα measurement by ELISA (Human TNFα DuoSet, R&D Systems, Abingdon, UK). When required, PBMCs were pre-incubated for 30 minutes prior to β-glucan training with 10 μM 3AC. Inhibitor was also added together with the first wash-out, before the resting period.

To assess cell viability, 3·10⁶ total PBMCs were plated in 24-well plates (1200-μl final volume, Corning) and followed the training scheme described here. At day 7, prior to LPS stimulation, cells were collected in PBS/EDTA and stained on ice-cold FACS Buffer for flow cytometry analysis.

For TNFα normalization, the fold of cells in each condition was calculated as follows: (Live cell number in condition X)/(live cell number in non-trained WT condition). In case of SHIP-1 inhibition experiments, non-treated cells were used as reference. Thus, TNFα per cell number was normalized as (absolute TNFα value)/(fold of cells).

### Example 6. In Vivo Models

Mice were trained with either two intraperitoneal (i.p.) injections of 1 mg β-glucan particles on days −7 and −4 or 2·10⁴ Candida albicans intravenously (i.v) on day −7. Sterile PBS was used as control. One week later, mice were challenged with 5 μg E. coli LPS (serotype 055:B5, Sigma) i.p. and blood was collected 60 min later to assess the serum TNFα (Mouse TNFα DuoSet, R&D Systems). Alternatively, mice were lethally infected with 2·10⁶ C. albicans i.v. and monitored daily for weight, general health and survival, following the institutional guidance. When required mice were i.p. treated with 0.11 mg 3AC on days −8 and −7.3AC was diluted in PBS 0.3% hydroxypropylcellulose (Sigma), used as control.

### Example 7. Western Blot

Cell lysates were prepared in RIPA buffer containing protease and phosphatase inhibitors (Roche, Basel, Switzerland). Samples were run on Mini-PROTEAN TGX PRECAST Gels and transferred onto a nitrocellulose membrane (both from Bio-Rad Laboratories, Hercules, Calif.) for blotting with the following antibodies: β-Actin (C4) and SHIP-1 (P1C1) from Santa Cruz (Dallas, Tex.); pAkt (Ser473, #4058S), Akt (#2920S), pS6 (Ser235/236, #4858T) and p4EBP1 (Thr37/46, #9459S), all from Cell Signaling (Danvers, Mass.). Alexa Fluor-680 (Life Technologies, Carlsbad, Calif.) or Qdot-800 (Rockland, Limerick, Pa.) conjugated secondary antibodies were used and gels were visualized in an Odyssey instrument (LI-COR, Lincoln, Nebr.).

### Example 8. Antibodies and Flow Cytometry

Samples were stained with the appropriate antibody cocktails in ice-cold FACS Buffer at 4° C. for 15 minutes. Antibodies included mouse PE-anti-TLR4 (BioLegend, San Diego, Calif.) and APC-anti-Dectin-1 (Bio-Rad). Dead cells were excluded by Hoechst 33258 (Invitrogen, Carlsbad, Calif.) incorporation. Purified anti-FcγRIII/II (2.4G2, TONBO Bioscience, San Diego, Calif.) was used to block murine Fc-receptors at 4° C. for 10 minutes in all the stainings. Events were acquired using FACSCanto 3L (BD Biosciences). Data were analyzed with FlowJo software (Tree Star, Ashland, Oreg.).

### Example 9. Glycolytic Flux Evaluation

The assay was performed in DMEM supplemented with 1 mM glutamine, 100 μg/ml penicillin, 100 μg/ml streptomycin. The pH was adjusted to 7.4 with KOH (herein called Seahorse medium). Cells were washed with PBS and 175 μl of Seahorse medium was added. Plates were incubated at 37° C. without CO₂ for 1h prior to the assay. Extracellular acidification rate (ECAR) was determined by using the glycolysis stress test in an XF-96 Extracellular Flux Analyzer (Agilent Technologies). Three consecutive measurements were performed under basal conditions and after sequential addition of 80 mM glucose (Merck), 904 oligomycin A (Sigma) and 500 mM 2-deoxy-glucose (2DG, Sigma). Basal and maximal glycolysis was defined as ECAR after addition of glucose and oligomycin, respectively. Glycolytic reserve was defined as the difference maximal and basal glycolysis.

### Example 10. Quantification and Statistical Analysis

The statistical analysis was performed using Prism software (GraphPad Software, La Jolla, Calif.). Statistical significance for comparison between two sample groups with a normal distribution (Shapiro-Wilk test for normality) was determined by two-tailed paired or unpaired Student's t test. When groups were too small to estimate normality, Gaussian distribution was assumed. Comparison of survival curves was carried out by Log-rank (Mantel-Cox) test. Outliers were identified by means of Tukey's range test Differences were considered significant at p<0.05 as indicated. Except when specified, only significant differences are shown. As indicated in figure legends, either a representative experiment or pool is shown or the number of repetitions of each experiment and number of experimental units (either cultures or mice) is indicated.

### Example 11. SHIP-1 Deletion Boosts Beta-Glucan-Induced Trained Immunity in Macrophages

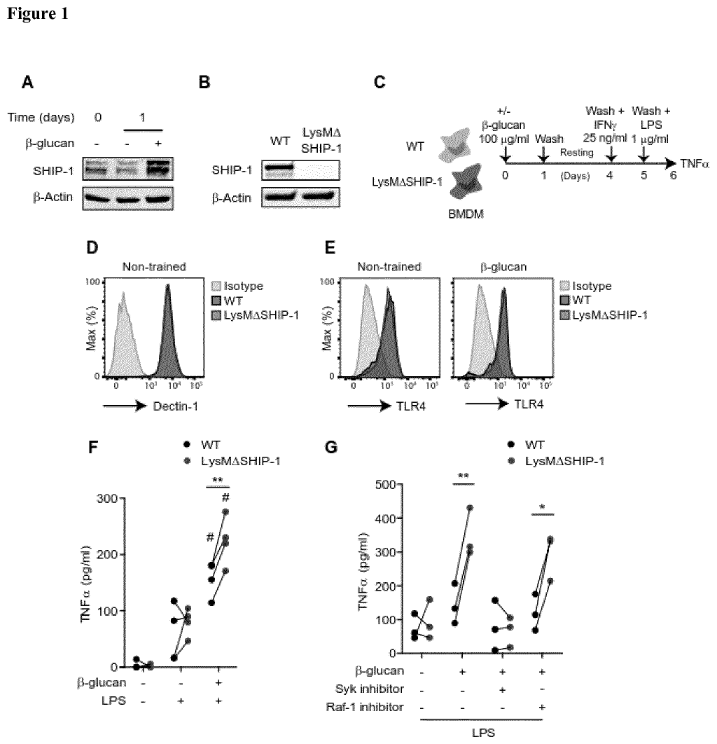

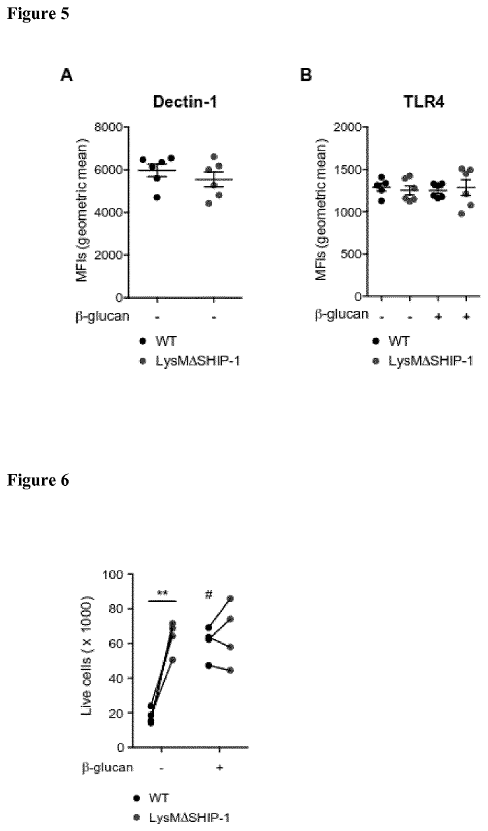

Dectin-1 sensing of beta-glucan induces trained immunity in human mononuclear phagocytes and PBMCs, purified mouse spleen monocytes and peritoneal or bone marrow-derived macrophages (BMDMs). We initially stimulated BMDMs with purified particulate beta-glucan from Saccharomyces cerevisiae, a well-known ligand for Dectin-1. The analysis of SHIP-1 protein in BMDMs by Western Blot (WB) revealed a basal expression that was further induced after 1 day of β-glucan training (FIG. 1A). To study the potential involvement of SHIP-1 in Dectin-1-triggered trained immunity, we generated BMDMs from wild-type (WT) mice or mice bearing a specific deletion of SHIP-1 in the myeloid compartment (LysMΔSHIP-1), which did not express SHIP-1 protein in BMDMs (FIG. 1B). Next, we adapted the proposed in vitro long-term scheme of trained immunity to IFN-gamma-primed BMDMs, evaluating whether training with β-glucan boosts TNFα production in response to LPS (FIG. 1C). Surface expression of the receptors involved in beta-glucan (Dectin-1, FIG. 1D and FIG. 5A) and LPS (TLR4, FIG. 1E and FIG. 5B) recognition were comparable between WT and LysMΔSHIP-1 BMDMs. We found that β-glucan-induced training resulted in increased cell viability in BMDMs (FIG. 6), concurring with previous results on mouse and human monocytes. Non-trained SHIP-1-deficient BMDMs showed higher viability than their WT counterparts, showing similar cell numbers after beta-glucan training (FIG. 6). Thus, to ensure the analysis of cell-intrinsic responses as described, whenever analyzing cytokine production data were normalized to the number of live cells present in each treatment. Pre-incubation of WT BMDMs with beta-glucan prompted a greater production of TNF-alpha in response to LPS (FIG. 1F), reproducing trained immunity. Notably, beta-glucan-trained LysMΔSHIP-1 BMDMs showed an increased production of TNF-alpha compared with trained WT BMDMs (FIG. 1F). Of note, SHIP-1 deletion did not impact on this inflammatory response under non-trained conditions. These data indicate that SHIP-1 modulates the extent of LPS-induced TNF-alpha production specifically during β-glucan training. At the molecular level, trained inflammatory responses triggered by beta-glucan rely on Raf-1 and are independent of Syk in human monocytes and human PBMCs. However, while Syk inhibition (R406) abolished the boost of trained immunity observed in the absence of SHIP-1 in BMDMs, Raf-1 inhibition did not affect this process (FIG. 1G). Our data indicate that in BMDMs, SHIP-1 adjusts the development of beta-glucan-induced trained immunity in a Syk-dependent manner.

### Example 12. SHIP-1 Regulates Molecular and Metabolic Hallmarks of Trained Immunity

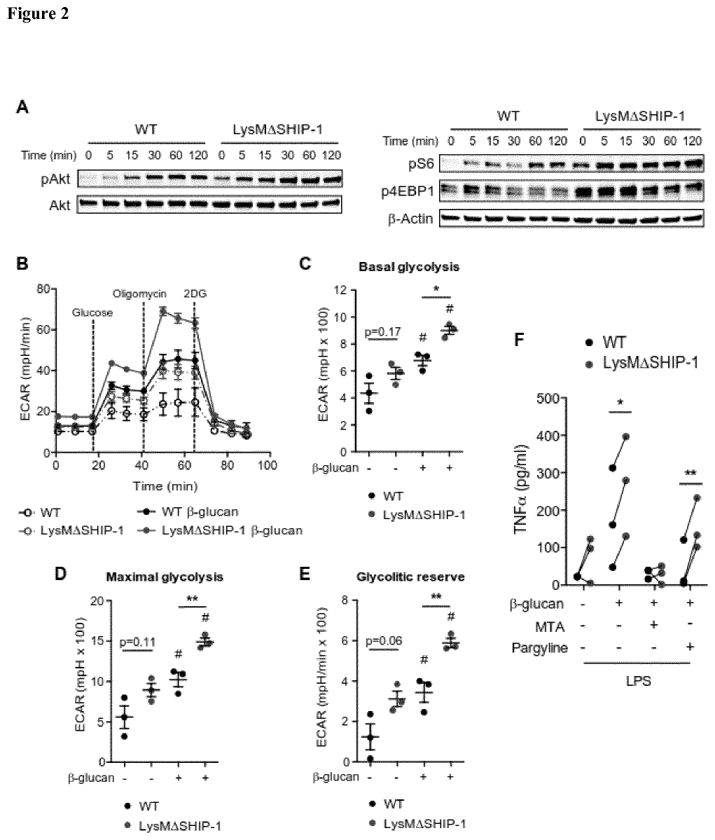

We tested whether the boost in β-glucan training in the absence of SHIP-1 in BMDMs was accompanied by regulation of additional hallmarks involved in the process. First, we monitored Akt activation after exposition of BMDMs to β-glucan. In WT cells, Akt was phosphorylated in response to Dectin-1 engagement in a time-dependent manner (FIG. 2A, left), concurring with previous results described in human monocytes. Notably, LysMΔSHIP-1 BMDMs showed increased and preserved Akt phosphorylation upon beta-glucan training (FIG. 2A, left). Then, we evaluated the activation of mTOR by analyzing the phosphorylation of two of its targets, S6 and 4EBP1, in response to β-glucan. In WT BMDMs, S6 phosphorylation was induced along the training time, while the activation of 4EBP1 was transient, peaking as soon as 5 minutes post challenge (FIG. 2A, right). Again, in the absence of SHIP-1, both targets displayed increased phosphorylation and maintained an over-activated state during the treatment with beta-glucan (FIG. 2A, right). Of note, a basal activation of the Akt/mTOR pathway occurs in LysMΔSHIP-1 BMDMs but it did not result in higher TNF-alpha production unless beta-glucan-induced trained immunity is established (FIG. 1F). Next, we measured the extracellular acidification rate (ECAR) in β-glucan trained BMDMs in a glycolysis stress test (FIG. 2B). Training with beta-glucan increased ECAR in WT BMDMs, a metabolic shift that was significantly boosted in trained SHIP-1-deficient BMDMs (FIG. 2B), as reflected by an enhanced basal (FIG. 2C) and maximal (FIG. 2D) glycolysis, together with a higher glycolytic reserve (FIG. 2E) in the absence of SHIP-1. These results suggest that SHIP-1 controls the extent of the glycolytic switch in BMDMs upon training with beta-glucan. Finally, we assessed whether the regulatory role of SHIP-1 on trained immunity relied on the epigenetic hallmarks induced by beta-glucan training. The histone demethylase inhibitor pargyline had no effect on the TNF-alpha overproduction observed in trained LysMΔSHIP-1 BMDMs (FIG. 2F). However, inhibition of histone methyltransferases using MTA inhibited the increase in TNF-alpha in the absence of SHIP-1 (FIG. 2F). These results highlight SHIP-1 as a regulator of trained immunity by dampening the Akt/mTOR molecular pathway and the glycolytic switch, and relying on the epigenetic reprogramming induced by beta-glucans, paradigms of the training process.

### Example 13. Myeloid-Specific Deletion of SHIP-1 Improves Trained Immunity In Vivo

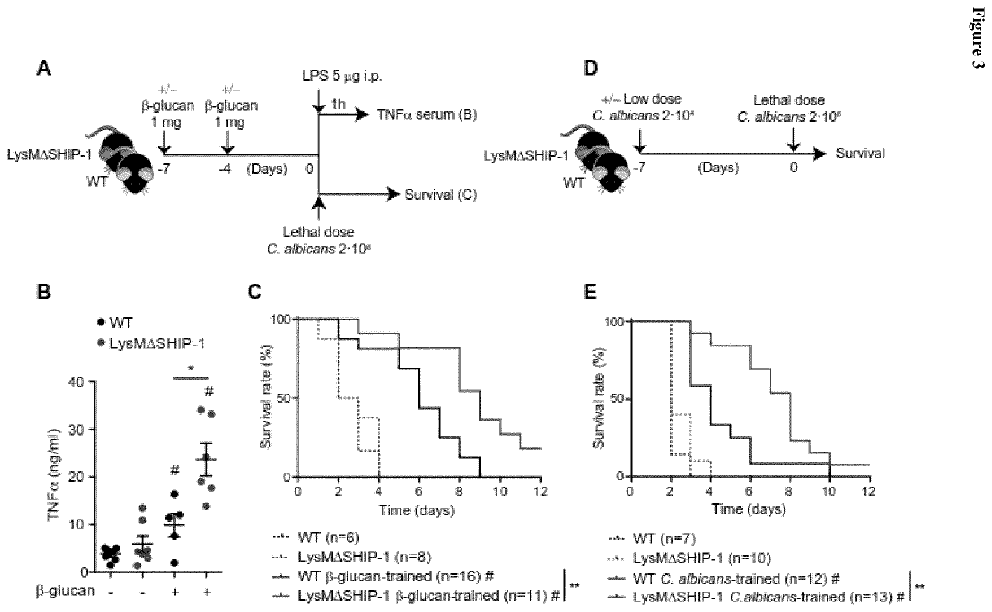

The generation of trained immunity in vivo leads to cross-protection against diverse secondary infections. Signaling through PI3K is the canonical molecular pathway implicated in the development of these trained responses. To test the role of myeloid SHIP-1 in cytokine production under beta-glucan training in vivo, WT and LysMΔSHIP-1 mice were challenged with LPS after two consecutive intraperitoneal injections of beta-glucan and TNFα was measured in serum 1h later (FIG. 3A). LPS-induced levels of TNF-alpha were increased in sera from WT mice receiving the beta-glucan pre-treatment (FIG. 3B), indicative of the generation of a trained response. Consistent with our results in vitro, this inflammatory response was exacerbated in LysMΔSHIP-1 trained-mice (FIG. 3B). Protective response against lethal systemic Candida albicans infection by trained immunity relies on monocytes and macrophages. After training with beta-glucan, WT and LysMΔSHIP-1 mice were intravenously infected with a lethal dose (2·10⁶) of the clinical isolate C. albicans SC5314 and survival was monitored (FIG. 3A). Both WT and LysMΔSHIP-1 non-trained mice rapidly succumbed upon these infectious conditions (FIG. 3C, dashed lines), indicating that SHIP-1 expression in the myeloid compartment is redundant for the primary response to lethal candidiasis. Beta-glucan administration trained WT mice against a lethal C. albicans infection, extending their lifespan (FIG. 3C, solid lines). Notably, LysMΔSHIP-1 mice improved beta-glucan-induced protection compared with WT animals (FIG. 3C, solid lines). As trained immunity can be defined as a protection mechanism from secondary lethal C. albicans infection induced by a nonlethal encounter with the same pathogen, we trained mice with a low dose (2·10⁴) of C. albicans followed by a lethal dose of the fungus (2·10⁶) seven days afterwards, and survival was monitored (FIG. 3D). Again, the training stimulus enlarged the survival time of WT mice (FIG. 3E, solid lines). Notably, LysMΔSHIP-1 trained mice were more resistant than WT to lethal systemic candidiasis (FIG. 3E, solid lines). These data indicate that SHIP-1 in myeloid cells dampens β-glucan and Candida-induced trained immunity in vivo, improving response to pathogen-specific or heterologous challenges.

### Example 14. Pharmacological Inhibition of SHIP-1 Enhances Trained Immunity

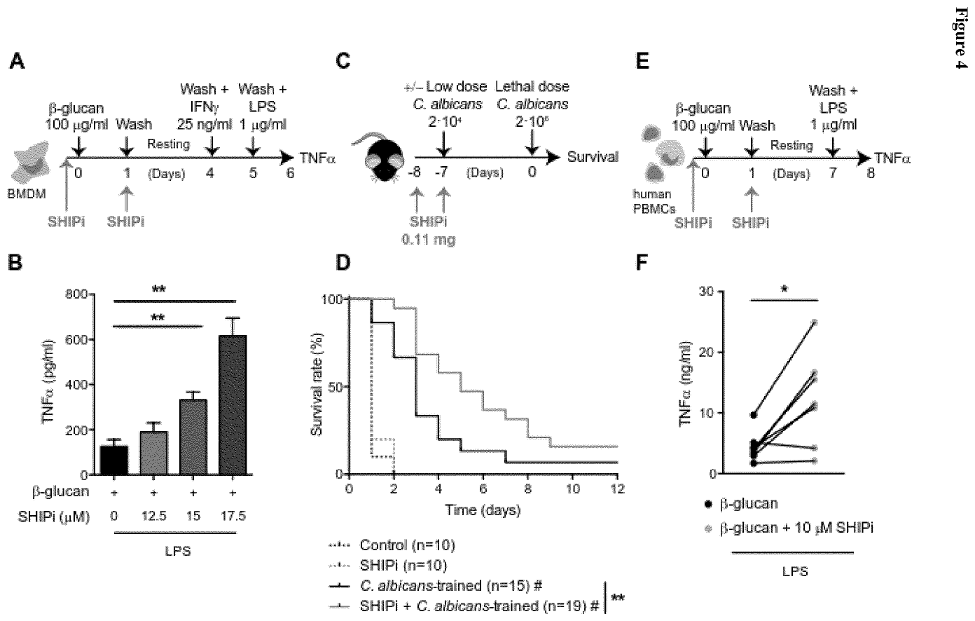

The relevance of the PI3K pathway has promoted the study of the phosphatase SHIP-1 as a potential therapeutic target. Indeed, 3-alpha-aminocholestane (3AC), a chemical SHIP-1 inhibitor (SHIPi) has been shown to promote T and NK cell control of tumors but also in therapies aimed to expand bone marrow precursors after radiotherapy. We thus tested 3AC as a potential tool to boost trained immunity. BMDMs were pre-exposed to different doses of 3AC (IC₅₀=13.5 μM) 30 minutes before training with beta-glucan and added again after washing beta-glucan out (FIG. 4A). The production of TNF-alpha was measured in supernatants of BMDMs after resting and challenge with LPS as above (FIG. 1C). Upon training with beta-glucan, SHIP-1 inhibition boosted TNF-alpha production in a dose-dependent manner (FIG. 4B). This measurement was only performed in beta-glucan-trained cells, as non-trained BMDMs did not survive the 5 day-long in vitro culture in the presence of 3AC, while the inhibitor did not affect survival of trained BMDMs. This result suggests that SHIP-1 pharmacological inhibition could be used to improve trained immunity. To analyze the effect of 3AC SHIPi under in vivo infectious conditions, mice were administered SHIPi twice in consecutive days following the published regimen (Gumbleton et al., 2017) and, coincident with the second day of 3AC administration, mice were trained with a low dose of C. albicans. Seven days later, mice were lethally infected with the same fungus and survival was examined (FIG. 4C). Inhibition of SHIP-1 did not impact on the survival of non-trained mice (FIG. 4D, dashed lines), but improved the survival of Candida-trained mice (FIG. 4D, solid lines), indicating that chemical inhibition of SHIP-1 boosts trained immunity in vivo. To further explore the potential relevance of the use of 3AC SHIPi, we exposed human PBMCs to SHIPi for 30 minutes and trained then with beta-glucan along one day. Afterwards, cells were washed out with SHIPi-containing medium and rested for 6 days. Then, cells were washed again, stimulated with LPS and TNF-alpha production was measured in the supernatants (FIG. 4E). As for mouse BMDMs, detection of TNF-alpha was only performed in beta-glucan-trained human PBMCs, as SHIPi was toxic for non-trained BMDM cells (not shown). Importantly, SHIP-1 inhibition boosted TNF-alpha production in these beta-glucan-trained human PBMCs (FIG. 4F). Thus, our data indicate that SHIP-1 can be targeted with pharmacological inhibitors both in mouse and human cells to boost trained immunity.

1. SHIP-1 inhibitor, characterized by the Formula (I), or any salt thereof, 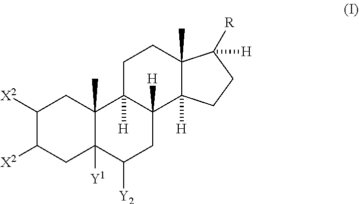
wherein,
X₁ is an amine,
X₂ can be H or OH or amine,
R is a C₁-C₁₁ alkyl,
Y₁ can be H or OH,
Y₂ can be H or OH,
or a molecule able to specifically target SHIP-1 gene and inhibit its translation,
for use in the non-specific prophylactic treatment or prevention of infectious diseases, wherein the SHIP-1 inhibitor, or the molecule able to specifically target SHIP-1 gene and inhibit its translation, is administered before, after or simultaneously to a treatment with a pathogenic microorganism or any part thereof which causes a stimulus responsible for training the innate immune cells.

2. SHIP-1 inhibitors for use, according to claim 1, wherein the SHIP-1 inhibitor is selected from:

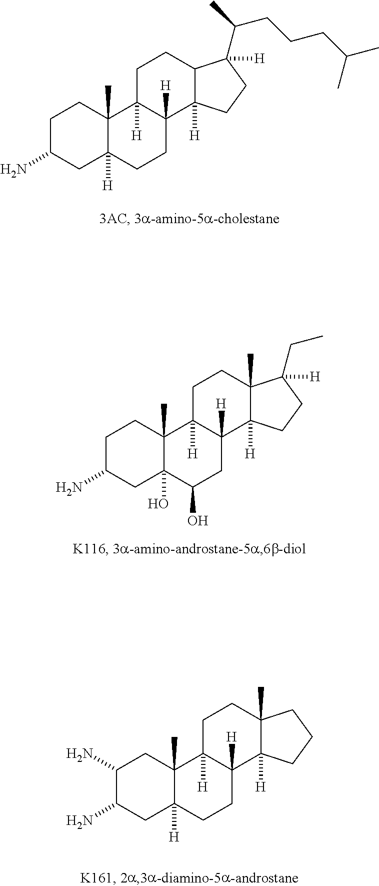

3. SHIP-1 inhibitor for use, according to any of the previous claims, characterized in that the SHIP-1 inhibitor is 3α-aminocholestane (3AC).

4. SHIP-1 inhibitors for use, according to any of the previous claims, in the non-specific prophylactic treatment or prevention of second or further infectious diseases caused either by the same or different microorganisms, wherein the SHIP-1 inhibitor is administered before, after or simultaneously to a treatment with a pathogenic microorganism or any part thereof responsible for training the innate immune cells.

5. SHIP-1 inhibitor for use, according to any of the previous claims, characterized in that it specifically targets SHIP-1 gene and inhibits its translation, or it is an antagonist selective for SHIP-1.

6. SHIP-1 inhibitor for use, according to any of the previous claims, wherein the infectious disease is caused by an infection with a Gram negative or Gram positive bacteria, viruses, fungi or parasites.

7. SHIP-1 inhibitor for use, according to any of the previous claims, wherein the pathogenic microorganism or any part thereof which causes a stimulus responsible for training the innate immune cells is beta-glucan or Candida albicans.

8. SHIP-1 inhibitor for use, according to any of the previous claims, wherein the beta-glucan is beta-1,3(d)-glucan, preferably derived from Saccharomyces cerevisiae.

9. Combination drug product comprising a SHIP-1 inhibitor characterized by the Formula (I), or any salt thereof, 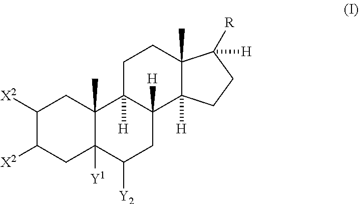
wherein,
X₁ is an amine,
X₂ can be H or OH or amine,
R is a C₁-C₁₁ alkyl,
Y₁ can be H or OH,
Y₂ can be H or OH,
or a molecule able to specifically target SHIP-1 gene and inhibit its translation,
a pathogenic microorganism or any part thereof which causes a stimulus responsible for training the innate immune cells and, optionally, pharmaceutically acceptable carriers.

10. Combination drug product, according to the claim 9, characterized in that the SHIP-1 is selected from:

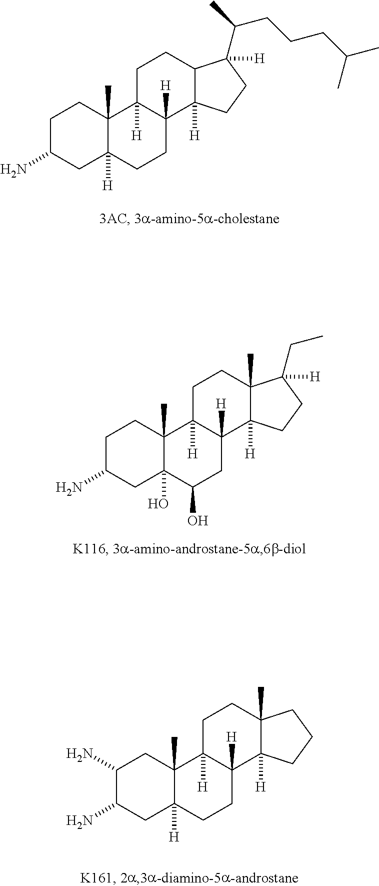

11. Combination drug product, according to any of the claim 9 or 10, characterized in that the SHIP-1 inhibitor is 3α-aminocholestane (3AC).

12. Combination drug product, according to any of the claims 9 to 11, wherein the pathogenic microorganism or any part thereof which causes a stimulus responsible for training the innate immune cells is beta-glucan or Candida albicans.

13. Pharmaceutical composition comprising the combination drug product according to any of the claims 9 to 12 and optionally pharmaceutically acceptable carriers.

14. Pharmaceutical composition according to claim 13, characterized in that it is a vaccine composition comprising the combination drug product.
